(19) United States
(12) Patent Application Publication
(10) Pub. No.: US 2004/0138483 A1
(43) Pub. Date: Jul. 15, 2004

(54) Olefin oxide catalysts

(76) Inventor: Leonid Isaakovich Rubinstein, Houston, TX (US)

Correspondence Address: Jennifer D. Adamson, Shell Oil Company, Intellectual Property Services, P. O. Box 2463, Houston, TX, 77252-2463, US

(21) Appl. No.: 10/694,312

(22) Filed: Oct. 27, 2003

Related U.S. Application Data
(60) Provisional application No. 60/421,753, filed on Oct. 28, 2002.

(51) Int. Cl. C07D 301/03

(52) U.S. Cl. 549/536

(57) ABSTRACT

The present invention provides an improved oxidation catalyst composition containing a catalytically effective amount of silver and a rubidium promoter deposited on a carrier, which rubidium metal promoter provides a quantity of rubidium at least 5 μmole and less than 60 μmole per gram of catalyst composition. The catalysts of the invention are deposited on carriers such as α-alumina and silver-bonded calcium carbonate. The invention is also directed to a process for the oxidation of olefins, which process involves reacting the olefin with oxygen in the presence of a catalyst composition having a catalytically effective amount of silver and a rubidium promoter deposited on a carrier, wherein said rubidium metal promoter provides a quantity of rubidium of at least 5 μmole and less than 60 μmole per gram of catalyst composition.

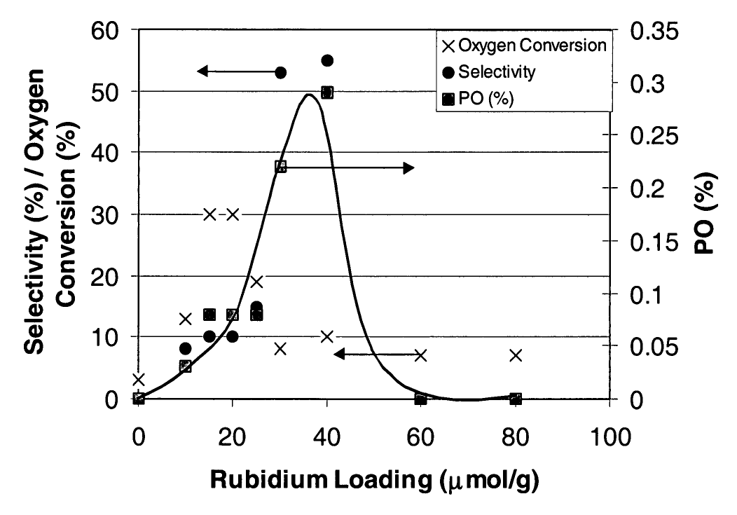

### FIELD OF THE INVENTION

The invention relates to silver containing supported catalysts comprising a promoter, and processes for the preparation of such catalysts. The invention also relates to processes for preparing olefin oxides by direct oxidation of olefins using oxygen-containing gases in the presence of a supported catalyst composition comprising silver and a promoter.

### BACKGROUND OF THE INVENTION

In olefin oxidations, catalyst performance may be assessed on the basis of selectivity, activity and stability of operation. The selectivity is the percentage of the olefin in the feed stream yielding the desired olefin oxide. As the catalyst ages, the percentage of the olefin conveted normally decreases. In order to maintain a constant level of olefin oxide production, the temperature of the reaction is increased. However, this adversely affects the selectivity of the conversion to the desired olefin oxide. Because the reactor equipment can withstand temperatures only up to a certain level, it is necessary to terminate the reaction when the temperature reaches an unacceptable level. Thus, the longer the selectivity can be maintained at a high level and the oxidation can be performed at an acceptable temperature, the longer the catalyst charge can be kept in the reactor and the more product is obtained. Quite modest improvements in the maintenance of selectivity over long periods potentially yield large dividends in terms of process efficiency. Catalysts and processes capable of producing olefin oxides by vapor phase direct oxidation in higher yields and selectivities than are presently attainable would therefore be desirable.

### SUMMARY OF THE INVENTION

The invention provides a catalyst composition comprising: a carrier; a catalytically effective amount of silver; and, a rubidium promoter in a quantity comprising from at least 5 μmole and less than 60 μmole per gram of catalyst composition.

The invention also provides a process for the oxidation of olefins which process comprises reacting the olefin with oxygen in the presence of a catalyst composition comprising silver and a rubidium promoter deposited on a carrier, wherein said rubidium metal promoter comprises a quantity of at least 5 μmole and less than 60 μmole per gram of catalyst composition.

### BRIEF DESCRIPTION OF THE FIGURE

The Figure shows the catalyst activity, selectivity, and oxygen conversion as a function of rubidium loading on the silver catalysts of the invention.

### DETAILED DESCRIPTION OF THE INVENTION

The invention is directed to a catalyst composition and a process for the oxidation of an olefin.

The catalysts of the present invention are olefin oxidation silver based catalysts containing rubidium. The catalysts of the invention comprise silver in an effective amount to impart catalytic activity, rubidium in an effective amount to lower the reaction temperature needed to achieve a certain production of olefin oxide and improve catalyst selectivity, and a carrier for said catalyst. The catalysts of the invention are particularly useful for preparing propylene oxide via silver catalyzed oxidation of propylene.

The quantity of silver which may be supported on the carrier may be selected within wide ranges. Suitably the quantity of silver is in the range of from 0.5% by weight to 60% by weight, preferably 0.75% by weight to 58% by weight and more preferably from 1% by weight to 55% by weight, relative to the weight of the catalyst composition.

The quantity of rubidium in the catalyst is at least 5 μmole and less than 60 μmol per gram of catalyst, preferably from 20 μmol per gram of catalyst to up to 60 μmol per gram of catalyst and more preferably from 30 μmol per gram of catalyst to 50 μmol per gram of catalyst.

The catalyst carrier may be based on a wide range of materials. Such materials may be natural or artificial inorganic materials and they may include refractory materials, silicon carbide, clays, zeolites, charcoal and alkaline earth metal carbonates, for example calcium or magnesium carbonate. Refractory materials that can be used include alumina, especially α-alumina, magnesia, zirconia and silica. Carriers based on α-alumina and a silver bonded calcium carbonate are particularly preferred.

Typically, the carrier is a porous carrier, having a specific surface area of from 0.01 m²/g to 50 m²/g, preferably from 0.05 m²/g to 30 m²/g, more preferably from 0.1 m²/g to 10 m²/g, still more preferably from 1.0 m²/g to 10 m²/g, as measured by the B.E.T. method, and an apparent porosity of from 0.05 ml/g to 3 ml/g, preferably from 0.1 ml/g to 2 ml/g, more particularly from 0.05 ml/g to 1 ml/g, still more preferably from 0.3 ml/g to 0.5 ml/g, as measured by conventional water absorption technique. The B.E.T. method as referred to herein has been described in detail in S. Brunauer, P. Y. Emmett and E. Teller, J. Am. Chem. Soc. 60, 309-16 (1938).

Of particular interest are α-aluminas which have a pecific surface area of from 0.1 m²/g to 25 m 2/g, preferably from 0.3 m²/g to 10 m²/g, as measured by the B.E.T. method, and which have an apparent porosity of from 0.1 ml/g to 0.6 ml/g, preferably from 0.1 ml/g to 0.55 ml/g, as measured by conventional water absorption technique. Particularly, these α-aluminas have a relatively uniform pore diameter. Specific examples of such α-aluminas are marketed by NorPro, under the trademark ALUNDUM®, and by Südchemie.

Also of particular interest are α-alumina carriers which can be used have a surface area of at least 1 m²/g, and a pore size distribution such that pores with diameters in the range of from 0.2 μm to 10 μm represent at least 70% of the total pore volume and such pores together provide a pore volume of at least 0.27 ml/g, relative to the weight of the carrier. Pore volume and pore size distribution can be measured by a conventional mercury intrusion device in which liquid mercury is forced into the pores of the carrier. Greater pressure is needed to force the mercury into the smaller pores and the measurement of pressure increments corresponds to volume increments in the pores penetrated and hence to the size of the pores in the incremental volume. The pore volumes in the instant application were determined by mercury intrusion under pressures increased by degrees to a pressure of 3.0×10⁸ Pa using a Micromeritics Autopore 9200 model (130° contact angle and mercury with a surface tension of 0.473 N/m).

Also of particular interest α-alumina carriers that can be used for supporting the catalysts of the invention are made from mixtures comprising: (a) from 50% w to 90% w, particularly from 65% w to 75% w, of a first particulate α-alumina having an average particle size (d₅₀) of from more than 10 μm up to 100 μm, particularly from 11 μm to 60 μm, more particularly from 12 μm to 40 μm; and (b) from 10% w to 50% w, particularly from 25% w to 35% w, of a second particulate α-alumina having a d₅₀ of from 1 μm to 10 μm, particularly from 2 μm to 6 μm; the % w being based on the total weight of α-alumina in the mixture.

The particulate α-alumina are readily commercially available, or they may readily be made, for example, by subjecting course materials to grinding and sieving operations. In an embodiment of the present invention, the smaller particles may be prepared from the larger particles by grinding, and the ground and un-ground particles may then be combined. In another embodiment, the desired mixture of large and small particles may be formed by grinding relatively large particles to the extent that the mixture of particles has the desired bimodal particle size distribution.

When the carrier is an α-alumina carrier, in particular, one comprising at least 80% w, or particularly at least 90% w, or more particularly at least 95% w α-alumina, it is preferred that the carrier include a coating material based on a silica-containing composition comprising a crystallization inhibitor, inhibiting the formation of crystalline silica-containing compositions, and also, preferably, providing a coating of a non-crystalline silica compound to the carrier surface. Preferably, the coating material also acts as a bond material for the α-alumina particles.

Typically, silica-containing compositions for use as a coating material comprise an amorphous silica compound which may be, for example, a silica sol, a precipitated silica, an amorphous silica, or an amorphous alkali metal silicate or alumosilicate. Typically, silica-containing compositions for use as a coating material may also comprise hydrated alumina. The crystallization inhibitor that is most conveniently incorporated is an alkali metal compound, in particular a water soluble salt, such as a sodium or potassium salt.

A convenient coating material may comprise a mixture of boehmite, ammonium silicate or silica sol, and a water soluble sodium salt. Similar effects can be achieved by incorporation of conventional ceramic bonds formulated to contain aluminosilicates and an alkali metal component.

A preferred coating material is based on (a) from 1% w to 10% w, particularly 2% w to 5% w, of an alumina hydrate, calculated as aluminium oxide relative to the weight of the α-alumina; (b) from 0.1% w to 1% w, particularly 0.2% w to 0.8% w, of an amorphous silica compound, as specified hereinbefore, calculated as silicon oxide relative to the weight of the α-alumina; and (c) from 0.01% w to 0.5% w, particularly 0.05% w to 0.3% w, of an alkali metal compound, calculated as the alkali metal oxide relative to the weight of the α-alumina.

In a preferred embodiment, the alumina carrier has an alumina content of at least 95% w and may be made by a method which comprises forming a mixture comprising: (a) from 65% w to 75% w, relative to the total weight of α-alumina in the mixture, of a first particulate α-alumina having a d₅₀ of from 10 μm to 60 μm, in particular from 12 μm to 40 μm; (b) from 25% w to 35% w, relative to the total weight of α-alumina in the mixture, of a second particulate α-alumina having a d₅₀ of from 2 μm to 6 μm; (c) from 2% w to 5% w of an alumina hydrate, calculated as aluminum oxide relative to the total weight of α-alumina in the mixture; (d) from 0.2% w to 0.8% w of an amorphous silica compound, as specified hereinbefore, calculated as silicon oxide relative to the total weight of α-alumina in the mixture; and (e) from 0.05% w to 0.3% w, of an alkali metal compound, calculated as the alkali metal oxide relative to the total weight of α-alumina in the mixture; and then forming the mixture into shaped bodies and firing the shaped bodies at a temperature of from 1050° C. to 1500° C. to form the carrier.

The preferred alumina hydrate is boehmite, though gibbsite, bayerite or diaspore may also be used.

Suitable alkali metals are, for example, lithium, sodium and potassium, or combination thereof. Suitable alkali metal compounds are, for example, alkali metal carbonates, alkali metal acetates, alkali metal formates, alkali metal nitrates, and combinations thereof. Typically, the overall atomic ratio of silicon to the alkali metal is in the range of from 1 to 10, particularly 2 to 8, and more particularly 6. The overall atomic ratio of silicon to the alkali metal is deemed to relate to the total alkali metal content and the total silicon content of the carrier, which includes any alkali metal and any silicon which may be present in the carrier other than in the bond material.

It is preferred that the carrier particles be prepared in the form of shaped bodies, the size of which is in general determined by the dimensions of a reactor in which they are to be deposited. Generally, however, it is found very convenient to use particles such as shaped bodies in the form of powdery particles, trapezoidal bodies, cylinders, saddles, spheres, doughnuts, and the like. The cylinders may be solid or hollow, straight or bent, and they may have the same length and cross-sectional dimensions, which may be from 5 mm to 10 mm. Particularly, for use in a tubular fixed bed reactor, they are formed into a rounded shape, for example in the form of spheres, pellets, cylinders, rings or tablets, typically having dimensions in the range of from 2 mm to 2 cm.

The shaped bodies can be formed from the mixture by any convenient molding process, such as spraying, spray drying, agglomeration or pressing, but particularly they are formed by extrusion of the mixture. For applicable methods, reference may be made to, for example, U.S. Pat. No. 5,145,824; U.S. Pat. No. 5,512,530; U.S. Pat. No. 5,384,302; U.S. Pat. 5,100,859; and U.S. Pat. No. 5,733,842, all of which are hereby incorporated by reference. To facilitate such molding processes, in particular extrusion, the mixture may suitably be compounded with up to 30% w and particularly from 2% w to 25% w, based on the weight of the mixture, of extrusion aids. Extrusion aids (also referred to by the term “processing aids”) are known in the art (cf., for example, “Kirk-Othmer Encyclopedia of Chemical Technology”, 4th edition, Volume 5, pp. 610 ff.). Suitable extrusion aids may be, for example, petroleum jelly, hydrogenated oil, synthetic alcohol, synthetic ester, glycol, polyolefin oxide or polyethylene glycol. Burnout materials are typically applied in a quantity of up to 30% w, in particular from 2% w to 25% w, relatively to the weight of the mixture. Boric acid may also be added to the mixture, for example in a quantity of up to 0.5% w, more typically in a quantity of from 0.01% w to 0.5% w. The effect of the presence of boric acid may be a reduced content of leachable alkali metal ions in the carrier after firing. Enough water may be added to the mixture to make the mixture extrudable (by the term “the weight of the mixture”, as used hereinbefore, is meant the weight of the total mixture, but excluding the weight of any added water).

The shaped bodies are then dried and fired at a temperature high enough to ensure that the alumina particles are joined together by a sintering action and/or by the formation of bond posts formed from the bond material, if incorporated in the mixture. Generally, drying may take place between 20° C. and 400° C., particularly 25° C. to 350° C. and most particularly between 30° C. and 300° C., typically for a period of up to 100 hours and particularly from 5 minutes to 50 hours. Typically, drying is performed to the extent that the mixture contains less than 2% w of water. Generally, firing may take place between 1050° C. and 1500° C., particularly between 1100° C. and 1470° C., more particularly between 1150° C. and 1450° C., typically for a period of up to 5 hours and particularly for from 2 to 4 hours. Drying and firing may be carried out in any atmosphere, such as in air, nitrogen, or helium, or mixtures thereof. Preferably, in particular when the shaped bodies contain organic material, the firing is at least in part or entirely carried out in an oxidizing atmosphere, such as in oxygen containing atmosphere.

It has been found that the performance of the catalyst may be enhanced if the carrier is washed, to remove soluble residues, before deposition of other catalyst ingredients on the carrier. On the other hand, unwashed carriers may also be used successfully. A useful method for washing the carrier comprises washing the carrier in a continuous fashion with hot, deionized water, until the electrical conductivity of the effluent water does not further decrease. A suitable temperature of the deionized water is in the range of 80° C. to 100° C., for example 90° C. or 95° C. Reference may be made to WO-00/15333, which is hereby incorporated by reference.

Another preferred carrier for the catalysts of the invention is a silver bonded calcium carbonate characterized by a high relative surface area. The silver bonded support is typically a composition having a minimum compressive crush strength of 22N (5 pounds), particularly at least 40 N (9 lbs), and more particularly at least 53N (12 lbs) comprising: a shaped calcium carbonate compound having been treated with a silver compound to form a paste, which is subsequently extruded, and then calcined to produce a shaped silver bonded calcium carbonate compound.

A preferred calcium carbonate for making the silver bonded supports is a calcium carbonate which has a specific surface area of from 1 m²/g to 20 m²/g, particularly from 2 m²/g to 18 m²/g, and more particularly from 3 m 2/g to 15 m²/g, as measured by the B.E.T. method, and which has an apparent porosity of from 0.05 ml/g to 2 ml/g, particularly from 0.07 ml/g to 1.8 ml/g, and more particularly from 0.1 ml/g to 1.5 ml/g, as measured by conventional water absorption technique.

A carrier of calcium carbonate preferably comprises 80-99% by weight calcium carbonate and 1-20% by weight of silver, particularly 85-97% by weight calcium carbonate and 3-15% by weight silver and most particularly 90-95% by weight calcium carbonate and 5-10% by weight silver. The silver bonded calcium carbonate carrier is made by mixing a commercially available calcium carbonate powder with an aqueous silver oxalate ethylenediamine complex, having a concentration of silver from 15-33% by weight, particularly 27-33% w, in such quantities that the final ratio of silver/calcium carbonate is approximately from 1:5 to 1:100, particularly from 1:6 to 1:30, more particularly from 1:8 to 1:10. and most particularly 1:9. After mixing the above components, an organic extrusion aide such as starch and optionally a burnout material is added to the mix, such that there are 90-100 parts by weight (pbw) calcium carbonate mixed with 1-2 pbw of the extrusion aid. Then, a sufficient amount of water, generally 35-45 pbw silver solution, is added to make the composition extrudable, and the resulting composition is mixed until homogeneous and extrudable. The resulting paste is then extruded. One method of extrusion may be to force the paste through a die of from 0.5 mm to 5 cm, particularly from 1 mm to 5 mm. The extrudate may then be fired at a temperature ranging from 180° C. to 870° C., particularly from 200° C. to 750° C. for 1-12 hours. The resulting extrudate may also first be dried over a period of 1 hour to 18 hours at for example from 10° C. to 500° C., particularly from 50° C. to 200° C., more particularly from 80° C. to 120° C. and then fired. An example of a program for firing the catalyst may be: an 0.1-10 hour ramp, such as 1 hour ramp, from 200° C. to 250° C., held for 1 hour, then a 4 hour ramp from to 500° C. and held for 5 hours. The resulting catalyst carrier has good mechanical properties, particularly crush strength, and is suitable to manufacture the catalysts of the invention useful for oxidation of olefins.

In a suitable method of catalyst preparation, the carrier is impregnated with a liquid composition of compounds of silver and rubidium or other useful additives, and subsequently dried by heating at a temperature in the range of from 150° C. to 500° C., particularly from 200° C. to 450° C., for a period of from 1 minute to 24 hours, particularly from 2 minutes to 2 hours, more particularly from 2 minutes to 30 minutes, in an atmosphere of air, an inert gas, such as nitrogen or argon, or steam.

Reducing agents will generally be present to effect the reduction of a silver compound to metallic silver. For example, a reducing atmosphere, such as a hydrogen containing gas, may be employed, or a reducing agent may be present in one or more of the impregnation liquids, for example, oxalate. If desired, the pore impregnation may be carried out in more than one impregnation and drying step. For example, silver may be impregnated in more than one step, and the promoters may be impregnated in one or more separate steps, prior to silver impregnation, after silver impregnation or intermediate to separate silver impregnation steps. The liquid composition is typically a solution, more typically an aqueous solution.

The compounds employed in the impregnation may independently be selected from, for example, inorganic and organic salts, hydroxides and complex compounds. They are employed in such a quantity that a catalyst is obtained of the desired composition.

The catalysts of the present invention are useful for oxidation of any olefin which has at least 2 carbon atoms. Typically the number of carbon atoms is at most 10, more typically at most 5. It is most preferred that the number of carbon atoms is three. The process comprises reacting an olefin with oxygen in the presence of a catalyst composition of the invention as described herein above.

By the term “improved catalyst performance” it is meant that there is an improvement in at least one catalyst property. Catalyst properties include, but are not limited to, catalyst activity, selectivity, activity or selectivity performance over time, operability (i.e. resistance to run-away), conversion, work rate and process temperatures. By “selectivity” is meant the selectivity to olefin oxide, based on the quantity of olefin converted.

The present process also provides a process for making derivatives of the instant propylene oxide, such as propylene glycol and polymers of propylene oxide. Any suitable process known to one skilled in the art for converting alkylene oxide to alkylene oxide derivatives can be utilized.

It also has been found that in the partial oxidation of higher olefins with oxygen an improved catalyst performance can be achieved by employing a supported silver catalyst which further comprises a rubidium metal promoter.

Apart from having an olefinic linkage (i.e. a moiety >C=C<), the olefin may comprise another olefinic linkage, or any other kind of unsaturation, such as an aryl group, for example, a phenyl group. Thus, the olefin may be a conjugated or non-conjugated diene or a conjugated or non-conjugated vinyl aromatic compound, for example 1,3-butadiene, 1,7-octadiene, styrene or 1,5-cyclooctadiene.

In preferred embodiments, the olefin comprises a single olefinic linkage and for the remainder it is a saturated hydrocarbon. It may be linear, branched or cyclic. A single alkyl group may be attached to the olefinic linkage, such as in 1-hexene, or two alkyl groups may be attached to the olefinic linkage, such as in 2-methyl-octene-1 or pentene-2. It is also possible that three or four alkyl groups are attached to the olefinic linkage. Two alkyl groups may be linked together with a chemical bond, so that together with the olefinic linkage they form a ring structure, such as in cyclohexene. In these preferred embodiments, a hydrogen atom is attached to the olefinic linkage at the places which are not occupied by an alkyl group. It is particularly preferred that a single alkyl group is attached to the olefinic linkage.

Preferred olefins having at least 3 carbon atoms are 1-pentene, 1-butene and, in particular, propylene. The skilled person will appreciate that depending on its geometry, an olefin may yield a mixture of olefin oxides, for example olefin oxides in more than one isomeric form.

Generally, the process of this invention is carried out as a gas phase process, which is a process wherein gaseous reactants are reacted under the influence of a solid catalyst of the invention. Frequently, the reactants and any further components fed to the process are mixed to form a mixture which is subsequently contacted with the catalyst. The ratio of the quantities of the reactants and the further components, if any, and the further reaction conditions are not material to this invention and they may be chosen within wide ranges. As, generally, the mixture contacted with the catalyst is gaseous, the concentrations of the quantities of the reactants and the further components, if any, are specified below as a fraction of the mixture in gaseous form.

The concentration of the olefin may suitably be at least 0.1% v, typically at least 0.5% v, and the concentration may suitably be at most 60% v, in particular at most 50% v. Preferably, the concentration of the olefin is in the range of from 1% v to 40% v. If the olefin is propylene, 1-butene or 1-pentene it is preferred that its concentration is in the range of from 1% v to 30% v, in particular from 2% v to 15% v.

The concentration of oxygen may suitably be at least 2% v, typically at least 4% v, and in practice, the concentration is frequently at most 20% v, in particular at most 15% v. If the olefin is propylene, 1-butene or 1-pentene it is preferred that the concentration of oxygen is in the range of from 6% v to 15% v, in particular from 8% v to 15% v. The source of oxygen may be air, but it is preferred that an oxygen containing gas which may be obtained by separation from air is used.

Organic chloride compounds may be added to the mixture to improve catalyst selectivity. Examples of such organic chloride compounds are alkyl chlorides and alkenyl chlorides. Methyl chloride, vinyl chloride, 1,2-dichloroethane and, in particular, ethyl chloride are preferred organic chloride compounds. In the oxidation of propylene, the organic chloride concentration may be at least 20 ppm by volume, more particularly at least 50 ppm by volume, and the concentration may be at most 2000 ppm by volume, in particular at most 1500 ppm by volume, wherein ppm by volume is calculated as the molar quantity of chlorine atoms in the total quantity of the reactant mixture. The performance of the catalyst of the present invention may be improved by adding to the mixture a nitrate or nitrite forming compound. A nitrate or nitrite forming compound is a compound which is capable, under the conditions at which it is contacted with the catalyst, of introducing nitrate or nitrite ions on to the catalyst. In general, the nitrate or nitrite ions tend to disappear from the catalyst during the process, in which case they need to be replenished. As a consequence, it is preferred to add the nitrate or nitrite forming compound continuously to the mixture, or in a discontinuous mode at least at the points in time that the need thereto arises. For the initial stage of the process it may be sufficient to add the nitrate or nitrite forming compound or nitrate or nitrite ions to the catalyst at the stage of catalyst preparation. Preferred nitrate or nitrite forming compounds are nitric oxide, nitrogen dioxide and/or dinitrogen tetraoxide. Alternatively, hydrazine, hydroxylamine, ammonia, nitromethane, nitropropane or other nitrogen containing compounds may be used. A mixture of nitrogen oxides is preferably used, which may be designated by the general formula NOₓ, wherein x is a number in the range of from 1 to 2, expressing the molar average atomic ratio of oxygen and nitrogen of the nitrogen oxides in the mixture.

For propylene oxidation, the nitrate or nitrite forming compound may suitably be used at a concentration of at least 10 ppm by volume, particularly at least 20 ppm by volume, and the concentration is typically at most 200 ppm by volume, particularly at most 150 ppm by volume, more particularly at most 80 ppm by volume, and most particularly at most 50 ppm by volume, on the same basis.

Carbon dioxide may or may not be present in the mixture. Carbon dioxide may reduce catalyst activity and selectivity and, thus, the yield of olefin oxide. Carbon dioxide may typically be present at a concentration of at most 35% v, in particular at most 20% v.

Furthermore, inert compounds such as nitrogen, argon or methane, may be present in the mixture. Methane is preferred as it improves the dissipation of the heat of reaction, without adversely effecting the selectivity and the conversion.

The process may preferably be carried out at a temperature of at least 150° C., in particular at least 200° C. Preferably the temperature is at most 320° C., in particular at most 300° C. The process may preferably be carried out at a pressure of at least 0.5 barg (i.e. bar gauge), in particular at least 1 barg. Preferably the pressure is at most 100 barg, in particular at most 50 barg.

In general, it is preferred to operate at a high oxygen concentration. However, in actual practice in order to remain outside the flammability limits of the mixture of reactants and any further components present therein, the concentration of oxygen has to be lowered as the concentration of the olefin is increased. The actual safe operating conditions depends along with the gas composition, also on individual plant conditions, such as temperature and pressure, and tube sizes. Therefore, in each individual plant a so-called flammability equation is used to determine the concentration of oxygen which may be used to approximate the allowable oxygen concentration with any concentration of the olefin.

When operating the process as a gas phase process using a packed bed reactor, the GHSV may preferably be at least 100 Nl/(l.h), in particular at least 200 Nl/(l.h). The GHSV may preferably be at most 30000 Nl/(l.h), in particular at most 15000 Nl/(l.h). The term “GHSV” stands for the Gas Hourly Space Velocity, which is the volumetric flow rate of the feed gas, which is herein defined at normal conditions (i.e. 0° C. and 1 bar absolute), divided by the volume of the catalyst bed.

### EXAMPLES

### Example 1

Preparation Of Silver-Amine-Oxalate Stock Solution

A silver-amine-oxalate stock solution was prepared by the following procedure: 415 g of reagent-grade sodium hydroxide were dissolved in 2340 ml de-ionized water and the temperature was adjusted to 50° C. 1699 g high purity “Spectropure” silver nitrate was dissolved in 2100 ml de-ionized water and the temperature was adjusted to 50° C. The sodium hydroxide solution was added slowly to the silver nitrate solution, with stirring, while maintaining a solution temperature of 50° C. This mixture was stirred for 15 minutes, then the temperature was lowered to 40° C. Water was removed from the precipitate created in the mixing step and the conductivity of the water, which contained sodium and nitrate ions, was measured. An amount of fresh deionized water equal to the amount removed was added back to the silver solution. The solution was stirred for 15 minutes at 40° C. The process was repeated until the conductivity of the water removed was less than 90 μmho/cm. 1500 ml fresh deionized water was then added.

630 g of high-purity oxalic acid dihydrate were added in approximately 100 g increments. The temperature was maintained at 40° C. and the pH was maintained at a level above 7.8. Water was removed from this mixture to leave a highly concentrated silver-containing slurry. The silver oxalate slurry was cooled to 30° C. Then 699 g of 92% w ethylenediamine (8% de-ionized water) were added to the slurry while maintaining a temperature no greater than 30° C. The resulting solution contained approximately 27-33% w silver.

### Example 2

General Procedure For Preparation Of Catalysts

Enough 45% w aqueous rubidium hydroxide and water was added to a solution prepared as in Example 1 to give a finished catalyst having 28% w silver, and rubidium loadings as specified in Table I.

The α-alumina carriers had a BET surface area of 2.0 m²/g, and an apparent porosity of 0.4 ml/g, measured by water absorption which contained 28 weight % silver, and a rubidium content which was varied from 0 μmol/g of rubidium to 80 μmol/g of rubidium.

An α-alumina carrier sample of approximately 30 g was placed under a 25 mm Hg vacuum for 1 minute at ambient temperature. Approximately 100 g of the impregnating solution was then introduced to submerse the carrier, and the vacuum was maintained at 25 mm Hg for an additional 3 minutes. The vacuum was then released and the excess impregnating solution was removed from the catalyst pre-cursor by centrifugation at 500 rpm for two minutes. The catalyst pre-cursor was then dried while being shaken at 250° C. for 5.5 minutes in a stream of air.

### Examples 3-11

Catalyst Preparation And Testing For Propylene Oxide

All the catalysts were prepared on an α-alumina carrier of Example 2. Each was tested at 1800 hr⁻¹ GHSV, with 12% O₂, 8% C₃H₆, 150 ppm C₂H₅Cl, and 100 ppm NOₓ in the feed. The results are shown in Table I.

Table I shows catalyst performance, measured as the selectivity and the work rate at the point in time that the selectivity had stabilized. The selectivity is calculated as the % mole of propylene oxide produced, relative to the propylene consumed. The work rate is the rate of propylene oxide production per unit weight of catalyst (kg/(m³.h)).

TABLE I

| EXAM- | | | O₂ | Selec- | | |
|---|---|---|---|---|---|---|
| PLE | Rb | | Conversion | tivity | PO | Workrate |
| No. | (μmol/g) | T (° C.) | (%) | (%) | (%) | (kg/m³/hr) |
| 3 | 0 | 210 | 3 | 0 | 0 | 0 |
| 4 | 10 | 200 | 13 | 8 | 0.03 | 1.5 |
| 5 | 15 | 200 | 30 | 10 | 0.08 | 4 |
| 6 | 20 | 200 | 30 | 10 | 0.08 | 4 |
| 7 | 25 | 200 | 19 | 15 | 0.08 | 4 |
| 8 | 30 | 200 | 8 | 53 | 0.22 | 10 |
| 9 | 40 | 200 | 10 | 55 | 0.29 | 14 |
| 10 | 60 | 240 | 7 | 0 | 0 | 0 |
| 11 | 80 | 240 | 7 | 0 | 0 | 0 |

The results in Table I indicate that the use of rubidium in the concentration range of the present invention provides silver catalysts with improved catalyst performance for making propylene oxide. An interesting feature of the rubidium system is a sensitivity to temperature increases. The performance of the rubidium bonded catalyst as measured by activity, selectivity, and oxygen conversion as a function of rubidium loading is shown in the Figure.

Clearly, the rubidium bonded propylene oxide catalysts provide an opportunity to significantly lower the reaction temperature while maintaining propylene oxide production and improving catalyst selectivity.

The catalysts of the present invention are useful in a variety of catalytic applications in which a reactant stream (gaseous or liquid) is contacted with a catalyst supported on a carrier at elevated temperatures. There are many such processes in the chemical industry but the present carrier has proved itself particularly suitable in the catalytic formation of alkylene oxide from a gas stream comprising propylene and oxygen. The utility of the present invention is however not so limited.

The instant application shows a detailed description of particular embodiments of the invention as described above. It is understood that all equivalent features are intended to be included within the claimed contents of this invention.

What is claimed is:

1. A catalyst composition comprising:
a carrier;
a catalytically effective amount of silver; and,
a rubidium promoter comprising a quantity of from 5 μmole to up to 60 μmole per gram of catalyst composition.

2. The catalyst composition of claim 1, wherein the carrier comprises an α-alumina having a BET surface area of from 0.01 m²/g to 50 m²/g, and an apparent porosity of from 0.1 ml/g to 2 ml/g, measured by water absorption.

3. The catalyst composition of claim 1, wherein the carrier comprises a silver bonded calcium carbonate having a crush strength of at least 22 N.

4. The catalyst composition of claim 1, wherein the carrier comprises a silver bonded calcium carbonate wherein the weight ratio of silver to calcium carbonate is from 1:5 to 1:100.

5. The catalyst composition of claim 1, wherein the carrier comprises a silver bonded calcium carbonate having a specific surface area of from 1 m²/g to 20 m²/g.

6. The catalyst composition of claim 1, wherein the carrier comprises a silver bonded calcium carbonate having a specific surface area of from 1 m²/g to 3 m²/g.

7. The catalyst composition of claim 1, wherein the carrier comprises a silver bonded calcium carbonate having an apparent porosity of from 0.05 ml/g to 2 ml/g.

8. The catalyst composition of claim 1, wherein the carrier comprises a silver bonded calcium carbonate having an apparent porosity of from 0.1 ml/g to 1.5 ml/g.

9. The catalyst composition of claim 1, wherein the carrier comprises at least 95% w α-alumina.

10. The catalyst composition of claim 9, wherein the α-alumina carrier has a pore size distribution within a total pore volume such that pores with diameters in the range of from 0.2 μm to 10 μm represent more than 75% of the total pore volume; pores with diameters greater than 10 μm represent less than 20% of the total pore volume; and pores with diameters less than 0.2 μm represent less than 10% of the total pore volume.

11. The catalyst composition of claim 9, wherein the α-alumina carrier has a pore size distribution such that pores with diameters in the range of from 0.2 μm to 10 μm represent more than 90% of the total pore volume; pores with diameters greater than 10 μm represent less than 10% of the total pore volume; and pores with diameters less than 0.2 μm represent less than 7% of the total pore volume.

12. The catalyst composition of claim 9, wherein the α-alumina carrier has a surface area of at most 2.9 m²/g.

13. The catalyst composition of claim 9, wherein the α-alumina carrier has a water absorption of at least 0.35 ml/g and a surface area in the range of from 1.4 m²/g to 2.6 m²/g.

14. The catalyst composition of claim 9, wherein the α-alumina carrier is made by a method which comprises:
forming a mixture comprising:
(a) from 50% w to 90% w of a first particulate α-alumina having an average particle size of from more than 10 μm up to 100 μm; and
(b) from 10% w to 50% w of a second particulate α-alumina having an average particle size of from 1 μm to 10 μm; the % w being based on the total weight of α-alumina in the mixture; and,
firing the mixture to form the carrier.

15. The catalyst composition of claim 14, wherein the α-alumina carrier comprises:
(a) from 65% w to 75% w, relative to the total weight of α-alumina in the mixture, of a first particulate α-alumina having an average particle size of from 11 μm to 60 μm;
(b) from 25% w to 35% w, relative to the total weight of α-alumina in the mixture, of a second particulate α-alumina having an average particle size of from 2 μm to 6 μm;
(c) from 2% w to 5% w of an alumina hydrate, calculated as aluminum oxide relative to the total weight of α-alumina in the mixture;
(d) from 0.2% w to 0.8% w of an amorphous silica compound, calculated as silicium oxide relative to the total weight of α-alumina in the mixture; and,
(e) from 0.05% w to 0.3% w of an alkali metal compound, calculated as the alkali metal oxide relative to the total weight of α-alumina in the mixture.

16. A process for the oxidation of an olefin, which process comprises reacting the olefin with oxygen in the presence of a catalyst composition comprising a carrier;
a catalytically effective amount of silver; and, a rubidium promoter, wherein the rubidium metal promoter comprises a quantity of from 5 μmole to up to 60 μmole per gram of catalyst composition.

17. The process of claim 16, wherein the carrier comprises a silver bonded calcium carbonate having a crush strength of at least 22 N.

18. The process of claim 16, wherein the carrier comprises a silver bonded calcium carbonate wherein the weight ratio of silver to calcium carbonate is 1:9.

19. The process of claim 16, wherein the carrier comprises a silver bonded calcium carbonate having a specific surface area of from 1 m²/g to 20 m²/g.

20. The process of claim 16, wherein the carrier comprises a silver bonded calcium carbonate having a specific surface area of from 1 m²/g to 3 m²/g.

21. The process of claim 16, wherein the carrier comprises a silver bonded calcium carbonate having an apparent porosity of from 0.05 ml/g to 2 ml/g.

22. The process of claim 16, wherein the carrier comprises a silver bonded calcium carbonate having an apparent porosity of from 0.1 ml/g to 1.5 ml/g.

23. The process of claim 16, wherein the carrier comprises an α-alumina carrier which has been obtained by a method which comprises:
forming a mixture comprising:
(a) from 50% w to 90% w of a first particulate α-alumina having an average particle size of from more than 10 μm up to 100 μm; and,
(b) from 10% w to 50% w of a second particulate α-alumina having an average particle size of from 1 μm to 10 μm; and wherein the % w is based on the total weight of α-alumina in the mixture;
forming the mixture into shaped bodies; and,
firing the shaped bodies to form the carrier.

24. The process of claim 16, wherein the carrier comprises an α-alumina carrier having a pore size distribution in a total pore volume such that pores with diameters in the range of from 0.2 μm to 10 μm represent more than 75% of the total pore volume; pores with diameters greater than 10 μm represent less than 20% of the total pore volume; and pores with diameters less than 0.2 μm represent less than 10% of the total pore volume.

25. The process of claim 16, wherein the carrier comprises an α-alumina carrier having a pore size distribution in a total pore volume such that pores with diameters in the range of from 0.2 μm to 10 μm comprise more than 90% of the total pore volume; pores with diameters greater than 10 μm represent less than 10% of the total pore volume; and pores with diameters less than 0.2 μm represent less than 7% of the total pore volume.

26. The process of claim 16, wherein the carrier comprises an α-alumina carrier having a surface area of at most 2.9 m²/g.

27. The process of claim 16, wherein the carrier comprises an α-alumina carrier having a water absorption of at least 0.35 ml/g and a surface area in the range of from 1.4 m²/g to 2.6 m²/g.

28. The process of claim 16, wherein the carrier comprises an α-alumina carrier made by a method which comprises:
forming a mixture comprising:
(a) from 50% w to 90% w of a first particulate α-alumina having an average particle size of from more than 10 μm up to 100 μm; and
(b) from 10% w to 50% w of a second particulate α-alumina having an average particle size of from 1 μm to 10 μm; the % w being based on the total weight of α-alumina in the mixture; and,
firing the mixture to form the carrier.

29. The process of claim 16, wherein the carrier comprises an α-alumina carrier having a composition comprising:
(a) from 65% w to 75% w, relative to the total weight of α-alumina in the mixture, of a first particulate α-alumina having an average particle size of from 11 μm to 60 μm;
(b) from 25% w to 35% w, relative to the total weight of α-alumina in the mixture, of a second particulate α-alumina having an average particle size of from 2% w to 6% w;
(c) from 2% w to 5% w of an alumina hydrate, calculated as aluminum oxide relative to the total weight of α-alumina in the mixture;
(d) from 0.2% w to 0.8% w of an amorphous silica compound, calculated as silicium oxide relative to the total weight of α-alumina in the mixture; and,
(e) from 0.05% w to 0.3% w of an alkali metal compound, calculated as the alkali metal oxide relative to the total weight of α-alumina in the mixture.

30. The process of claim 16, which process further comprises adding an organic chloride promoter.

31. The process of claim 30, wherein the organic chloride promoter is present at a concentration of at least 50 ppm by volume.

32. The process of claim 16, which process further comprises adding a NOₓ promoter, wherein x is 1 or 2.

33. The process of claim 32, wherein the NOₓ promoter is present at a concentration of 500 ppm by volume.

34. A composition comprising propylene oxide, made by a process comprising reacting propylene with oxygen in the presence of a catalyst composition comprising silver and a rubidium promoter deposited on a carrier, wherein the rubidium metal promoter comprises a quantity of from 30 μmole to 50 μmole per gram of catalyst composition

35. A composition comprising a derivative of propylene oxide, wherein the propylene oxide is made by a process comprising reacting propylene with oxygen in the presence of a catalyst composition comprising a carrier; a catalytically effective amount of silver; and, a rubidium promoter deposited on a carrier, wherein the rubidium metal promoter comprises a quantity of from 5 μmole to up to 60 μmole per gram of catalyst composition.
